(19) United States
(12) Patent Application Publication
(10) Pub. No.: US 2014/0328911 A1
(43) Pub. Date: Nov. 6, 2014

(54) COMPOSITIONS COMPRISING POMEGRANATE POLYPHENOLS IN COMBINATION WITH ERECTILE DYSFUNCTION-TREATING COMPOUNDS, AND METHODS OF TREATING ERECTILE DYSFUNCTION THEREWITH

(71) Applicant: Bradley K. Gillespie, Reston, VA (US)

(72) Inventor: Bradley K. Gillespie, Reston, VA (US)

(73) Assignee: POM WONDERFUL. LLC, Los Angeies, CA (US)

(21) Appl. No.: 14/364,612

(22) PCT Filed: Jul. 19, 2012
(86) PCT No.: PCT/US12/47461

§ 371 Date: Jun. 11, 2014

Related U.S. Application Data
(60) Provisional application No. 61/510,837, filed on Jul. 22, 2011.

(51) Int. Cl. A61K 31/7048; A61K 31/366; A61K 31/519; A61K 31/53; A61K 31/4985

(52) U.S. Cl. CPC A61K 31/7048; A61K 31/53; A61K 31/4985; A61K 31/519; A61K 31/366
USPC 424/463; 514/25

(57) ABSTRACT

The invention relates to compositions comprising a mixture of a pomegranate extract and an erectile dysfunction-treating compound. These compositions can be used to treat erectile dysfunction in men and sexual dysfunction in women. The pomegranate extract and the erectile dysfunction-treating compounds surprisingly and unexpectedly act in a synergistic manner, allowing the compositions to contain a lower amount of the pomegranate extract, the erectile dysfunction-treating compound, or both. The synergism of the two components of the composition results in a lower incident of side effects, as well as being less expensive. The invention also relates to products comprising a series of dosages of the compositions of the invention, wherein either the pomegranate extract or erectile dysfunction-treating compound can be raised or lowered, until a maintenance dosage level of the pomegranate extract and the erectile dysfunction-treating compound is reached.

### INTRODUCTION

The invention relates to compositions comprising a mixture of pomegranate extract and an erectile dysfunction-treating compound. These compositions can be used to treat erectile dysfunction in men and sexual dysfunction in women. The pomegranate extract, which contains polyphenolic compounds, and the erectile dysfunction-treating compounds surprisingly and unexpectedly act in at least an additive manner, and preferably in a synergistic manner, allowing the compositions to contain a lower amount of the pomegranate extract, the erectile dysfunction-treating compound, or both. The synergism of the two components of the composition results in a lower incident of side effects and lower cost. The invention also relates to products comprising a series of dosages of the compositions of the invention, wherein either the pomegranate extract or erectile dysfunctions drug can be raised or lowered, until a maintenance dosage level of the pomegranate extract and the erectile dysfunction-treating compound is reached.

### BACKGROUND OF THE INVENTION

Fruits and vegetables are an essential part of a healthy diet. One primary reason is that fruits and vegetables are rich sources of important phytochemicals. Phytochemicals provide essential nutrients and enhance the body's ability to prevent and fight disease. Among the wide variety of phytochemicals found in various fruits and vegetables, many exhibit a variety of beneficial activities including, but not limited to, antioxidant, anti-allergenic, anti-carcinogenic, anti-inflammatory, anti-viral, and/or anti-proliferative activities.

One common group of phytochemicals are antioxidants. Antioxidants are produced by the body or derived from food and have numerous beneficial effects on the body. When the body's natural antioxidant defenses are lowered or greater amounts of free radicals are being produced, the body becomes more dependent upon food sources of antioxidants. Antioxidants such as vitamin E and vitamin C have been used widely in clinical practice to protect the body from harmful free radicals.

Phytochemical antioxidants are found in a wide variety of plants, most notably in pomegranates. Pomegranates contain compounds with potent free radical scavenging capacities, including polyphenols. The polyphenol compounds having high antioxidant properties include water soluble tannins and proanthocyanins, among others. Polyphenols and other antioxidants are effective for protecting the cardiovascular system and are active against oxidative stress. For example, researchers have found that the consumption of pomegranate juice obtained from the arils of pomegranates has antioxidant properties by significantly inhibiting oxidative stress in mice and humans, as well as protecting against atherogenesis and atherosclerotic lesion development.

Oxidative stress is a major contributor to cardiovascular diseases and can cause tissue injury associated with lipid peroxidation in arterial macrophages and in lipoproteins. Such tissue injury occurs when the oxidative burden of the body exceeds its antioxidant capacity. Many researchers believe that the mechanism of oxidative injury involves lipid peroxidation, protein oxidation, DNA oxidation, decreased synthesis and bioavailability of endothelial (e) and neuronal (n) nitric oxide (NO), and the up-regulation of proinflammatory cytokines, growth factors and tissue specific receptors. Oxidative injury is also known to alter tissue structure and function in many organs, including the heart, blood vessels, lung, kidney and brain.

In addition to cardiovascular disease, oxidative injury or stress can also be involved in erectile dysfunction (ED). The physiologic mechanism of penile erection involves the release of nitric oxide (NO) into the corpus cavernosum during sexual stimulation. NO then activates guanylate cyclase, which increases cyclic guanosine momophosphate (cGMP). cGMP relaxes smooth muscle in the corpus cavernosum, allowing for blood inflow and penile erection. A reduction in NO due to oxidative stress interferes with this cascade and ultimately reduces or prevents penile erection.

In addition, other factors, such as vascular risk factors (e.g., hypercholesterolemia, atherosclerosis, hypertension, and diabetes mellitus) can interfere with the intricate neurovascular mechanisms underlying normal erection. Hypoxemia, sleep apnea, and respiratory failure are also increasingly recognized as causes of erectile dysfunction. These conditions are known to induce oxidative tissue injury due to the accumulation of reactive oxygen species (ROS) such as superoxide, H₂O₂, and hydroxyl radicals.

Pomegranate juice and extracts can exhibit potent antioxidant activity and can reduce or prevent oxidative tissue injury. Because ED has been linked to oxidative tissue injury, the juice and extracts have been administered to animals and humans in several experiments. Such administration has been shown to significantly shorten the time to full erection in an animal model of erectile dysfunction, because each increased prostatic blood flow and neurogenic smooth muscle relaxation (thought to be mediated by nitric oxide (NO)), while preventing erectile tissue fibrosis.

Thus, experimental studies using both pomegranate juice and pomegranate extracts (e.g., POM Wonderful pomegranate extract or “pomegranate extract”) should be continued in light of their effect on erectile dysfunction.

In one example, researchers analyzed the effect of pomegranate juice on erectile dysfunction. See Azadzoi K. M, et al. Oxidative stress in arteriogenic erectile dysfunction: prophylactic role of antioxidants, J. Urol., 174:386-93 (2005). The researchers employed pomegranate juice and in a rabbit model of arteriogenic erectile dysfunction (balloon injury of iliac arteries), where animals were given pomegranate juice (equivalent to 112 μmol polyphenols daily) for 8 weeks and showed reduced levels of oxidative stress in cavernous tissue relative to control. Further, pomegranate juice was shown to prevent fibrosis of erectile tissue in diseased animals and cause a significant increase in smooth muscle relaxation resulting in partial normalization of the differences between the ED and control groups. Similarly, while pomegranate juice was unable to completely normalize intracavernosal blood flow in the ED rabbits, the time to maximum intracavernosal pressure (time to achieve full erection) was significantly shorter in the pomegranate juice treated animals.

Researchers also found that pomegranate juice has the additional ability to increase vascular eNOS expression as well as levels of plasma nitrate and nitrite. Researchers hypothesized that these increases are due to both enhanced NO (widely believed to be the key neurotransmitter for penile erection), see Ignarro L. J., et al., Nitric oxide and cyclic GMP formation upon electrical field stimulation cause relaxation of corpus cavernosum smooth muscle, Biochem. Biophys. Res. Commun. 170:843-50 (1990), and production and decreases in its degradation, see Ignarro, L. J. et al., Pomegranate juice protects nitric oxide against oxidative destruction and enhances the biological actions of nitric oxide, Nitric Oxide 15:93-102 (2006).

In light of these experiments, an additional published human pilot study of 61 men with mild to moderate ED was performed. As described in the study, subjects were given 8 ounces of pomegranate juice and placebo per day for 28 days, each, in a crossover design study. See Forest C. P. et al., Efficacy and safety of pomegranate juice on improvement of erectile dysfunction in male patients with mild to moderate erectile dysfunction: a randomized, placebo-controlled, double-blind, crossover study, Int. J. Impot. Res. 19:564-567 (2007). Erectile function was measured using the Global Assessment Questionnaire (GAQ), a global patient assessment at the end of the observation period. A secondary endpoint was the International Index of Erectile Function (IIEF), which measures self-assessed erection function and satisfaction throughout the course of the observation period. Importantly, no serious adverse events occurred during the study, and no subjects discontinued due to adverse events. Although a statistically significant difference between treatment groups was not obtained (believed to be due to carryover effects and a small sample size), there was a noticeable trend towards an improvement in GAQ scores after treatment with pomegranate juice compared to placebo. An unfortunate confounding factor was a sequence effect; although the cohorts shared similar demographic and baseline characteristics, a larger difference in GAQ scores between pomegranate juice and placebo was observed in subjects who received pomegranate juice in the first treatment sequence. However, these investigators failed to explain, or speculate, on the cause of this sequence effect. Additionally, the investigators identified three key limitations to this study: relatively small sample size, short treatment duration (4 weeks), and lack of compliance. The researchers speculated that the use of a sexual encounter diary measure might have been a more sensitive measure of improvement in erectile function over the course of the study.

Other studies have employed a pomegranate extract. For example, rabbits were administered a liquid form of pomegranate extract for 8 weeks after arterial ballooning. A total of 20 animals were assigned to the ED group, while 20 age-matched animals were in the control group. Within these groups, animals were further divided by dose administered. An equal number of animals in each group were administered either placebo or one of three doses of liquid pomegranate extract (1×: 30 mg polyphenols, daily; 2×: 60 mg polyphenols daily or 4×: 120 mg polyphenols daily). ED animals showed a significant upregulation of oxidative stress sensitive genes encoding superoxide dismutase and aldose reductase. Treatment with pomegranate extract was able to reverse this upregulation to control levels. Further, pomegranate extract was able to normalize isoprostane 8-epi prostaglandin F2α (an oxidatively modified product associated with increased levels of oxidative stress) levels in the ED group to control levels. Electron microscopy showed that pomegranate extract was highly effective in preserving mitochondrial and endothelial structural integrity and diminishing caveolae on cell membranes in ED rabbits. Histomorphometric analysis of penile sections showed that pomegranate extract was able to limit the decrease in smooth muscle and limit the increase in fibrosis associated with ED relative to control. Although a dose response relationship was not observed, pomegranate extract was able to significantly increase the endothelium-dependent relaxation of erectile tissue and intracavernosal blood flow. The time to full erection in ED animals receiving pomegranate extract (1×: 15.2 seconds; 2×: 12.0; 4×: 17 seconds) was significantly shorter than the 28 seconds observed in the rabbits receiving placebo. See Zhang Q. et al., Dietary antioxidants improve arteriogenic erectile dysfunction. Int J Androl. (2010).

Both pomegranate juice and pomegranate extract have favorable safety profiles. Positive pre-clinical data and promising results were obtained in experiments using both pomegranate juice and pomegranate extract. These data suggest that pomegranate extract can be an effective treatment for men with ED.

Other methods of treating erectile dysfunction are also known. For example, erectile dysfunction-treating pharmaceutical compounds have now been on the market for many years. Such compounds include phophodiesterase-5 (PDE-5) inhibitors. PDE-5 is an enzyme that causes degradation of cGMP. By administration of the PDE-5 inhibitors, cGMP is increased, allowing for the cascade leading to penile erection. Commonly prescribed PDE-5 inhibitors include Levitra® (vardenafil), Clalis® (tadalafil), and Viagra® (sildenafil). Each of the three erectile dysfunction-treating compounds has a similar range of side effects, including headaches, flushing, dyspepsia, nasal congestion, urinary tract infection, abnormal vision, diarrhea, dizziness, rash and back pain. Other more serious side effects include loss of hearing, priapism, and respiratory infection.

While pomegranate extract and PDE-5 inhibitors are available to treat erectile dysfunction, these compounds have not been tested in combination. And there is still a need for a treatment that allows for lower drug administration and lower costs to those in need of such treatment. The instant invention provides for at least an enhanced, and preferably a synergistic combination of pomegranate extract and known erectile dysfunction-treating compounds, which in turn allows the administration of a lower therapeutically effective dose, as well as a lower cost therapy for those in need of treatment.

### SUMMARY OF THE INVENTION

One embodiment of the invention relates to pharmaceutical composition comprising a combination of a therapeutically effective amount of a pomegranate extract and a therapeutically effective amount of an erectile dysfunction-treating compound. The pharmaceutical composition can be further comprised of at least one pharmaceutically acceptable excipient. The combination of the pomegranate extract and erectile dysfunction-treating compound can be synergistic.

Another embodiment of the invention relates to a method of treating erectile dysfunction comprising administering to a subject a pharmaceutical composition comprising a combination of a therapeutically effective amount of a pomegranate extract and a therapeutically effective amount of an erectile dysfunction-treating compound. The pharmaceutical composition can be further comprised of at least one pharmaceutically acceptable excipient. The combination of the pomegranate extract and erectile dysfunction-treating compound can be synergistic.

In still another embodiment, the pomegranate extract can contain between about 20% to about 40% (by weight of the composition) of a combination of punicalin and punicalagin; and between about 1% to about 5% by weight of ellagic acid. The punicalagin can comprise the isomers punicalagin-α and punicalagin-β. Punicalagin-α can be present in an amount of about 20% to about 25% by weight of the composition and punicalagin-β can be present in an amount of about 4% to about 5%. The composition can also comprise other pomegranate polyphenol compounds, including, but not limited to, gallic acid, an isoflavone, which can be, but is not limited to genistein or daidzein, an anthocyanin, a hydrolyzable tannin, and/or combinations thereof. The isoflavone can be, but is not limited to, genistein, daidzein, methylated derivatives thereof, and glycoside derivatives thereof.

In another embodiment, the pomegranate extract can be prepared by the methods discussed below, or by any method that allows for the highest level of polyphenol extraction from the pomegranates, including extractions using water, alcohols, or other solvents. The amount of a solid form of the pomegranate extract to be administered can be between about 1,000 mg to about 2,000 mg or about 1,500 mg by weight. Because of the enhanced, or synergistic, activity between the pomegranate extract and the erectile dysfunction-treating compound, however, the dosage of the pomegranate extract can be reduced to about 500 mg to about 1,000 mg per dose by weight.

In one embodiment, the erectile dysfunction-treating compound can be, but is not limited to vardenafil, tadalafil, and sildenafil. The composition of the invention can comprise vardenafil in the approved dosages, which range from about 2.5 mg to about 20 mg (i.e., 2.5 mg, 5 mg, 10 mg, and 20 mg dosage forms). The maximum recommended dosing frequency is once per day about 60 minutes before sexual activity. Because of the enhanced, or synergistic, activity between vardenafil and the pomegranate extract, however, the dosage of vardenafil can be reduced to about 625 μg to about 2.5 mg per dose.

The composition of the invention can comprise tadalafil in the approved dosages, which range from about 2.5 mg to about 20 mg (i.e., 2.5 mg, 5 mg, 10 mg, and 20 mg dosage forms). Tadalafil has been approved by the FDA for once daily use. Because of the enhanced, or synergistic, activity between tadalafil and the pomegranate extract, however the dosage of tadalafil can be reduced to about 625 μg to about 2.5 mg per dose. The preferred combination is a reduced dosage version of the daily use tadalafil with the pomegranate extract that may be then used daily with even fewer side effects and at lower cost.

The composition of the invention can comprise sildenafil in the approved dosages, which range from about 20 mg to about 100 mg (i.e. 20 mg, 25 mg, 50 mg, and 100 mg dosage forms). The maximum recommended dosing frequency is once per day about 60 minutes before sexual activity. Because of the enhanced, or synergistic, activity between sildenafil and the pomegranate extract, however, the dosage of sildenafil can be reduced to about 5 mg to about 20 mg per dose.

In another embodiment, the invention relates to a package comprising a predetermined number of doses of a pharmaceutical composition comprising a combination, which can be synergistic, of a therapeutically effective amount of a pomegranate extract and a therapeutically effective amount of an erectile dysfunction-treating compound. The package can contain 28 doses of the pharmaceutical composition. The package itself can be in the form of a blister pack, blister card or blister envelope (collectively also known as Push-Through-Packs or “PTPs”), dial packs, dose packs, or any other form of packaging that can be used to contain approximately one month of doses, which allows for easier use and better patient compliance. The package can be further comprised of instructions for administration and use. The pharmaceutical composition in the package can be further comprised of at least one pharmaceutically acceptable excipient.

### BRIEF DESCRIPTION OF THE FIGURES

The invention can be better understood by references to the detailed description when considered in connection with the accompanying Figures.

FIGS. 1A-1E illustrate the variety of oral dosage forms that may be used with the present invention. FIG. 1A illustrates an embodiment of a round-shaped tablet. FIG. 1B illustrates an embodiment of a bilayer tablet. FIG. 1C illustrates an embodiment of a capsule. FIG. 1D illustrates an embodiment of a gelcap. FIG. 1E illustrates an embodiment of an oval-shaped tablet.

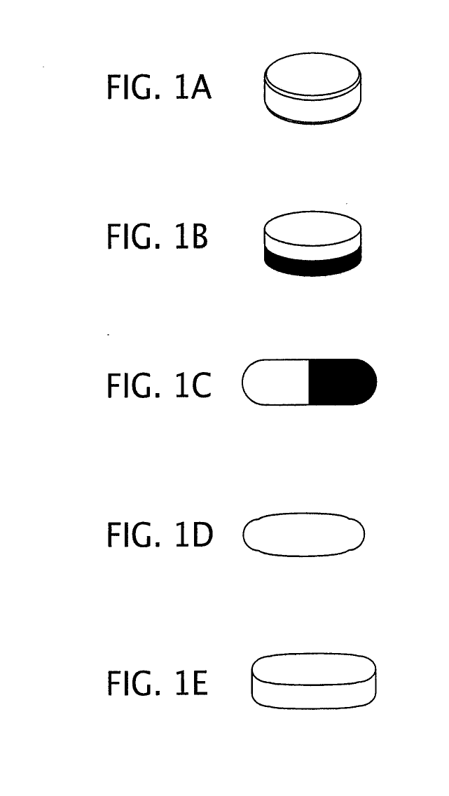

FIGS. 2A-2B depicts two potential blister packaging concepts that may be used in the present invention. FIG. 2A depicts a potential blister packaging concept wherein the pomegranate extract and the erectile dysfunction-treating compound are present in the same dosage form. FIG. 2B depicts a potential blister packaging concept wherein the pomegranate extract and the erectile dysfunction-treating compound are present in different dosage forms, but can be taken together.

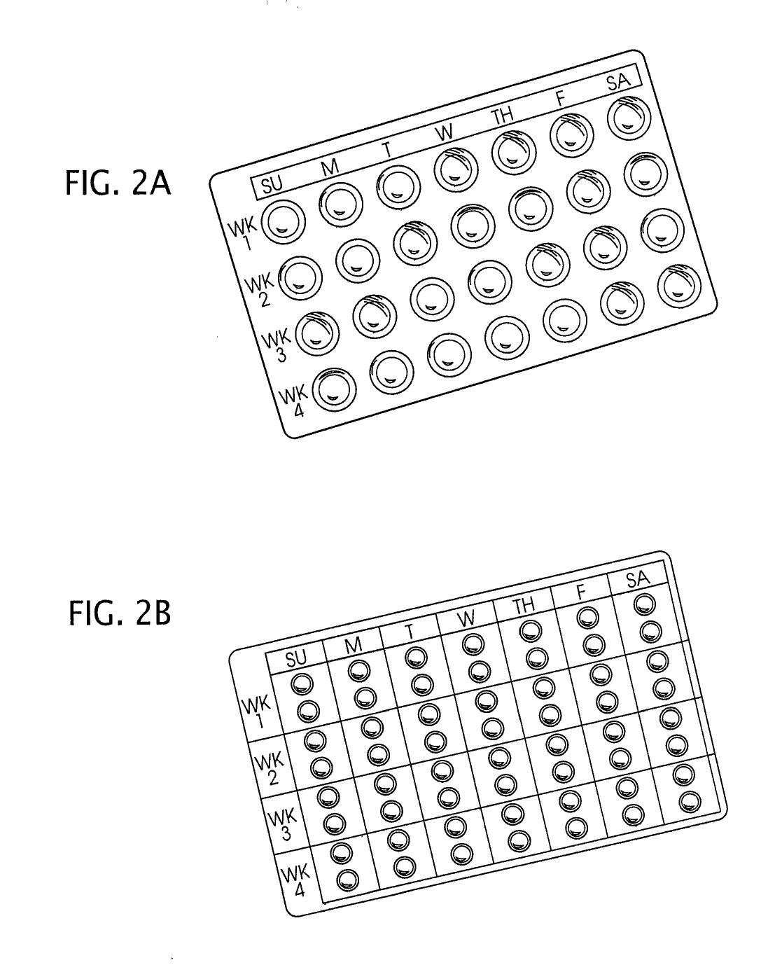

FIGS. 3A-3B depict a kit having a starter dosage and maintenance dosage packs in accord with the present invention. FIG. 3A depicts a kit having a starter dosage pack (weeks 1-4) and a maintenance dosage pack (weeks 5-8). FIG. 3B depicts a later maintenance dosage pack (weeks W-Z), which can be inserted into a kit holder if desired.

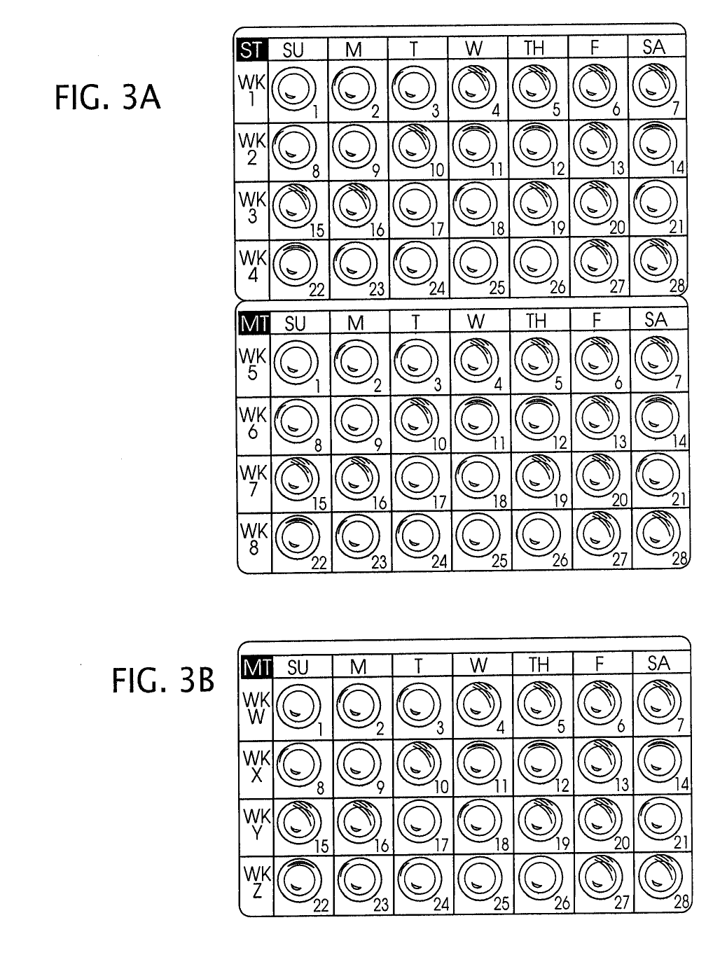

### DETAILED DESCRIPTION OF THE INVENTION

While the present disclosure can be embodied in many different forms, the drawings and discussions are presented with the understanding that the present disclosure is an exemplification of the principles of one or more inventions and is not intended to limit any one of the inventions to the embodiments illustrated.

As used herein, the term “phytochemicals” refers collectively to compounds which are naturally-occurring in the pomegranate and to reaction products and metabolites of these compounds, which are considered to have a beneficial effect on the human health. Examples of such phytochemicals include, but are not limited to polyphenols, estrogens, and phytoestrogens.

As used herein, the term “polyphenols” refers generally to a family of naturally-occurring compounds in the pomegranate and includes phenols and polyphenols. Phenols are a class of chemical compounds consisting of a single phenol unit in their structure. Although similar to alcohols, phenols have unique properties including relatively higher acidities due to the aromatic ring tightly coupled to the oxygen and a relatively loose bond between the oxygen and the hydrogen. Examples of phenolic compounds within this group include ellagic acid and gallic acid. Polyphenols are a group of compounds, characterized by the presence of more than one phenolic group. Polyphenols include, but are not limited to, tannins (e.g., ellagitannins and gallotannins), flavonoids (e.g., anthocyanins and isoflavones), punicalagin, punicalin, and stilbenes (e.g., resveratrol).

As used herein, the term “pomegranate extract” refers to a composition isolated from pomegranates that contains one or more of the polyphenol compounds discussed in paragraph [0031].

As used herein, the term “pomegranate juice” refers to the juice that is substantially obtained from the arils of the pomegranate.

As used herein, the term “pomegranate solids” refers to any one or a combination of the pericarp, the inner membrane and seeds of a pomegranate.

As used herein, the term “erectile dysfunction-treating compound” refers to a pharmaceutical compound that has the ability to improve or prevent difficulties in achieving penile erection. Further, these compounds can also have the ability to be used to treat sexual dysfunction in women. An exemplary class of such compounds includes phosphodiesterase-5 inhibitors, such as vardenafil, tadalafil, and sildenafil.

As used herein, the term “synergistic” or “synergistically” means that the therapeutic effect of administering the combination of the pomegranate extract and the erectile dysfunction-treating compound is greater than the additive effect of administering the pomegranate extract and the erectile dysfunction-treating compound individually. Such effects include one or more of increasing the effect of the pomegranate extract and/or erectile dysfunction-treating compound, increasing the duration of the effect of the pomegranate extract and/or the erectile dysfunction-treating compound, making the pomegranate extract and/or the erectile dysfunction-treating compound effective at dosage levels that would otherwise be ineffective for treating erectile dysfunction. The synergism between the pomegranate extract and the erectile dysfunction-treating compound allows for reduction of the amounts of the pomegranate extract or the erectile dysfunction-treating compound required to be therapeutically effective, which may minimize the occurrence of side effects and may effectively reduce the cost of therapy. The combination causes a greater effect than simply the sum of the individual effects of each drug if they were used separately.

The term “dosage unit” or “dose,” as used herein, refers to physically discrete units suitable as unitary dosages for animals, each unit containing a predetermined quantity of active material calculated to produce the desired therapeutic effect in association with the required diluents, e.g., a carrier or vehicle. The specifications for the unit dose of this invention are dictated by and are directly dependent on (a) the unique characteristics of the active material and (b) the limitations inherent in the art of compounding such active material for therapeutic use in animals.

The term “therapeutically effective amount,” as used herein, means that the amount of the extract of the present invention contained in the composition administered is of sufficient quantity to achieve the intended purpose, such as, in this case, as a prophylactic tool for preserving erectile tissue function and preventing cavernous fibrosis in arteriogenic ED in a subject.

Accordingly, one aspect of the present invention provides a method for treating a subject with ED. The method comprises the step of administering to the subject a composition comprising a therapeutically effective amount of an extract from pomegranate fruit in combination with an erectile dysfunction-treating compound. The combination of the pomegranate extract and the erectile dysfunction-treating compound can act in a synergistic manner. The extract of pomegranate can be a juice extract of pomegranate, an extract from inner or outer peel of pomegranate, a combination of pomegranate solids (including the pericarp, inner membrane and seeds) or the mixture thereof. The erectile dysfunction-treating compound can be, but is not limited to, vardenafil, tadalafil, or sildenafil.

One embodiment of the invention relates to pharmaceutical composition comprising a synergistic combination of a therapeutically effective amount of a pomegranate extract and a therapeutically effective amount of an erectile dysfunction-treating compound. The pharmaceutical composition can be further comprised of at least one pharmaceutically acceptable excipient.

Another embodiment of the invention relates to a method of treating erectile dysfunction comprising administering to a subject a pharmaceutical composition comprising a synergistic combination of a therapeutically effective amount of a pomegranate extract and a therapeutically effective amount of an erectile dysfunction-treating compound. The pharmaceutical composition can be further comprised of at least one pharmaceutically acceptable excipient.

The pomegranate extract in the administered composition can be between about 20% to about 40% (by weight of the composition) of a combination of punicalin and punicalagin; and between about 1% to about 5% by weight of ellagic acid. The punicalagin can comprise the isomers punicalagin-α and punicalagin-β. Punicalagin-α can be present in an amount of about 20% to about 25% by weight of the composition and punicalagin-β can be present in an amount of about 4% to about 5%. The composition can also comprise other pomegranate polyphenol compounds, including, but not limited to, gallic acid, an isoflavone, an anthocyanin, a hydrolyzable tannin, and/or combinations thereof. In addition, the pomegranate extract can further comprise up to a trace amount of anthocyanins. The trace amount of anthocyanins can be about 1% anthocyanins by weight, but depending on the method of manufacture can be as little as about 0.1% anthocyanins by weight.

Table 1 below exemplifies three different pomegranate extract compositions. One of ordinary skill in the art, however, would understand that the compositions described in Table 1 are exemplary only, and that the invention is not limited to the listed compositions or to a specific method of manufacture.

TABLE 1

| | | | Pomegranate | Pomegranate | Pomegranate |
|---|---|---|---|---|---|
| | | | Extract 1 | Extract 2 | Extract 3 |
| Component | | Method Type | Nominal (% by weight) | | |
| Total Hydrolyzable Tannins | | HPLC, UV-vis | | | |
| | Gallic acid (free) | | 0.05 to 0.15 | 0.8 to 1.2 | 0.8 to 1.2 |
| | Ellagic acid (free) | | 0.1 to 0.3 | 1.5 to 3.5 | 1.5 to 2.5 |
| | Gallic acid (hydrolyzed) | | | | |
| | Ellagic acid (hydrolyzed) | | | | |
| Total Punicalagin A&B | | HPLC, UV-vis | | | |
| | Punicalagin-α | | 0.80 to 0.95 | 1.0 to 2.0 | 5.5 to 6.5 |
| | Punicalagin-β | | 2.6 to 2.9 | 3.5 to 6.0 | 15 to 16 |
| | Punicalagin-α & -β | | 3.0 to 4.0 | 4.5 to 8.0 | 20 to 22 |

Surprisingly, the inventors discovered that the combination of the pomegranate extract and the erectile dysfunction-treating compound act in at least an additive manner, and preferably synergistically. Therefore, the amount of the erectile dysfunction-treating compound required for therapeutic efficacy can be lower in combination with the pomegranate extract than alone. In addition, the amount of the pomegranate extract required for therapeutic efficacy can lower in combination with the erectile dysfunction-treating compound than alone. Further, the amounts of the pomegranate extract and erectile dysfunction-treating compound required for therapeutic efficacy can both be lower in combination than alone.

As discussed above, researchers found that pomegranate juice has the additional ability to increase vascular eNOS expression, as well as levels of plasma nitrate and nitrite. It is also known that the class of therapeutic to which vardenafil, tadalafil, and sildenafil belong inhibit phosphodiesterase-5 (PDE-5) activity, which are able to inhibit the degradation of NO. Increases in eNOS expression are due to both enhanced NO production and decreased degradation. Thus, the inventors hypothesized that a treatment that allows for both increased eNOS expression and the inhibition of enzymes that inhibit PDE-5 (and thus inhibit degradation of NO) would exhibit at least an additive, and preferably synergistic, effect on erectile dysfunction.

The pharmaceutical composition can be administered in a variety of ways. For example, the composition can be administered orally, by injection, transdermally, buccally, or topically. The pharmaceutical composition can also be in a variety of forms. For example, the composition can be, but is not limited to, an immediate release composition, a delayed-release composition, an extended-release composition, a mixed-release composition, or an enterically-coated composition. The composition, if mixed-release, can be a bilayer tablet, with one half being an immediate release form, and the other layer being a delayed or extended release form. Generally, the composition can be administered to a subject in a capsule, a table, a suspension, an implant, a solution, an emulsion, a powder, a syrup, a liquid, an ointment, a lotion, a cream, a paste, or a gel. A dosage unit may comprise a solid or semisolid form. The solid or semisolid dosage form can have a weight between 0.1 and 30 grams, preferably between 0.2 and 10 grams. A daily dosage of the prepared polyphenol can include one or more pills, tablets or other dosage forms. Concentrated liquid forms are also contemplated and may be made by mixing in the powder or other forms of the polyphenol composition described herein. Alternatively or in addition the liquid concentrate itself can be produced from byproducts of the juice production process. In one embodiment the pomegranate extract and the erectile dysfunction-treating compound can be administered as separate compositions.

In another embodiment, the invention relates to a package comprising a predetermined number of doses of a pharmaceutical composition comprising a synergistic combination of a therapeutically effective amount of a pomegranate extract and a therapeutically effective amount of an erectile dysfunction-treating compound. The package itself can be in the form of a blister pack, blister card or blister envelope (collectively also known as Push-Through-Packs or “PTPs”), dial packs, dose packs, or any other form of packaging that can be used to contain approximately one month of doses, which allows for easier use and better patient compliance. The package can be further comprised of instructions for administration and use. The pharmaceutical composition in the package can be further comprised of at least one pharmaceutically acceptable excipient.

Subjects in the need of treatment for erectile dysfunction can need a period of acclimatization. For example, a subject in need of treatment for erectile dysfunction can need to adjust the amount of either component of the composition over the first month of administration to obtain the full effect of the composition, or to lower the dose to the lowest therapeutic amount of either the pomegranate extract or the erectile-dysfunction-treating compound. For example, the dosage of the erectile dysfunction-treating compound can be reduced over the 28 doses. Alternatively, the dosage of the erectile dysfunction treating compound can be increased over the 28 doses. Because the composition contains the synergistic combination of the pomegranate extract, a “starter” package can be initially administered to a subject. After the subject has reached the desired level of dosage for one or both of the composition component, “maintenance” packages can be prescribed for the subject. The “maintenance” package can have the pomegranate extract and the erectile dysfunction-treating compound remaining the same over the 28 doses. Each package can contain instructions for the administration of the pharmaceutical composition.

The starter package can be manufactured in several forms. As described above, a PTP can be manufactured so that the dosage of either the pomegranate extract or the erectile dysfunction-treating compound is found in a lower (or higher) dose with each proceeding week, with the dosage form differing each week, or even each day. Another way to present the starter package can include a PTP formation containing the predetermined doses of the pomegranate extract. The erectile dysfunction-treating compound can be associated with the PTP formation comprising the pomegranate extract, and can have a series of dosages to be taken in a prescribed manner, depending upon the time point in the starter pack. The erectile dysfunction-treating compound can also be in the PTP, in a separate PTP, or can be in a separate container portion of a kit, including all of the predetermined doses of the erectile dysfunction-treating compound. For example, the first week of the starter pack can provide a single daily pomegranate extract dose and a series of the smallest available dosage of the erectile dysfunction-treating compound. The erectile dysfunction-treating compound can be dosed at the full dose (or as many unit dosages to make up the full dose) for the first week. The second week, the pomegranate extract is taken from the PTP, in conjunction with a lower dose (or number of doses) of the associated erectile dysfunction-treating compound. The third week, the dosage of the erectile dysfunction-treating compound can be further reduced, with the fourth week requiring the lowest dose of the erectile dysfunction-treating compound.

If a patient determines that that a lower dose is not sufficient, the lowest effective dose can be determined and administered as the maintenance dose. Alternatively, the dosage of the pomegranate extract can be lowered during the starter phase, or both the pomegranate extract and the erectile dysfunction-treating compound can be lowered over the administration of the starter pack, as long as the lowest effective amount of each component of the composition is reached and administered during the maintenance phase.

The invention further relates to methods of treating erectile dysfunction with the pharmaceutical compositions and packages of compositions as described above. In addition, the amount of the pomegranate extract in the packaged pharmaceutical composition doses can be varied in the same way as the erectile dysfunction-treating compound. Alternatively, both portions of the composition can be reduced or increased until a maintenance dose is reached.

Turning to pomegranate extracts, it was been surprisingly discovered that extracts obtained from the pomegranate solids, in accordance with the methods disclosed herein, have substantially higher total polyphenol content than is found in the juice from the pomegranate arils. This is particularly true with respect to the higher molecular weight polyphenols (e.g., punicalagin).

Punicalagin is a powerful antioxidant, which is found in at least two forms, known as tautomers. The tautomers are punicalagin-α and punicalagin-β. Two features of punicalagins relate to the protection of cardiovascular function and accurate cellular replication. Thus, the punicalagin tautomers are responsible, in part, for the high antioxidant activity of the extract.

It is known that punicalagin can be hydrolyzed into ellagic acid. Once punicalagin hydrolyzes into ellagic acid, its ability to offer antioxidant potency to the body is reduced, since free ellagic acid is not as bioavailable. When punicalagins are preserved in their original, unhydrolyzed forms and then consumed, they can be absorbed into the bloodstream, and greater health benefits can be obtained. Punicalagins are 100% water-soluble, highly bioavailable, and shown to possess a high absorption rate up to 95%. Not only do punicalagins offer antioxidant activity on their own, they can break up into smaller polyphenols that are also absorbed into the body. Punicalagins are one important component of pomegranate polyphenols, but the total composition of the polyphenols themselves is a complex mixture of numerous other components.

While the antioxidant and other beneficial health effects of the extract are due to the presence of polyphenols, the presence of other phytochemical compounds in the extract, or the synergistic effect of these phytochemicals, can also be responsible for the antioxidant and other beneficial health effects of the extract. The reported soluble polyphenol content in pomegranate juice varies within the limits of approximately 0.2% to approximately 1.5%, and ellagic acid was measured in commercial juices around 100 to around 3000 mg/L.

In addition to punicalagin, other high molecular weight polyphenols have been characterized in the extract of pomegranate solids. These high molecular weight polyphenols include ellagitannin and other hydrolysable tannins, such as punicacortein A, punicalin, pedunculagin, and gallotanin dimers and trimers.

Another group of compounds found in pomegranates includes anthocyanins. A number of anthocyanins have been characterized in the extract of the pomegranate solids. Examples of the anthocyanins include pelargonidin 3-glucoside, cyaniding 3-glucoside, delphinidin 3-glucoside, pelargonidin 3,5-diglucoside, cyaniding 3,5-diglucoside, and delphinidin 3,5-diglucoside. Although these anthocyanins have been characterized in both the pomegranate juice and the extract, these lower molecular weight polyphenols comprise a higher proportion of the total polyphenol content in pomegranate juice, than in the extract.

Given that phenolic compounds found in pomegranates can be extracted from the fruit. Accordingly, methods are provided for producing an extract containing polyphenolic phytochemicals from pomegranate solids. The extract produced from the methods disclosed herein differ from the commercially-available pomegranate juice in that the extract is substantially derived from the pomegranate solids, whereas pomegranate juice is substantially derived from the sweet, fleshy arils that surround the pomegranate seed. The extract made by these methods is characterized as containing polyphenols and, particularly, high molecular weight polyphenols, such as punicalagin.

One method of making the pomegranate extract for use in treating ED in a synergistic combination with an erectile dysfunction-treating compound comprises selecting one or a combination of pomegranate solids, including the pericarp, inner membrane and seeds, and creating a mixture in an aqueous solution. The mixture of the pomegranate solids is created by adding water in an amount that is about 20 to about 80% w/v, preferably about 50% w/v, of the pomegranate solids. The mixture is crushed or milled to create a rough grind of pomegranate solids dispersed in the aqueous solution.

The mixture can then be heated to a temperature of about 60° F. to about 210° F., preferably at about 85° F. to about 185° F. and optimally of about 110° F. to about 160° F. The temperature to which the mixture is heated depends upon the selection of enzymes, or combination of enzymes, that is added to the mixture. Preferably, the mixture is heated to a temperature that permits the maximum catalysis of the enzyme or combination of enzymes.

Alternatively, enzymes can be added to the mixture before heating to assist in partially degrading the pomegranate solids. Thus, the order of the steps of heating the mixture and adding the enzymes is not critical, so long as the mixture is heated to a temperature that permits the enzymes to at least partially degrade the pomegranate solids. Heating can liberate the phytochemicals, which can then react and/or polymerize to create new phytochemical compounds or reaction products.

Enzymes suitable for use include those which are capable of at least partially degrading the plant tissue or cells to liberate the phytochemicals from the pomegranate solids. Such enzymes include, but are not limited to, any one or a combination of pectinase, cellulase, hemicellulase, amylase, arabanase, and other hydrolyzing enzymes. The enzymes added to the mixture can be naturally-occurring or synthetic. Such enzymes can be derived from any one or a combination of sources, including but not limited to, animal, plant, fungal, and bacterial sources. The amount of the enzyme or combination of enzymes added to the mixture depends on the temperature of the mixture and the amount of pomegranate solids present in the mixture.

After enzymes are added, the mixture can be maintained at a temperature for a time sufficient to allow at least partial degradation of the pomegranate solids. The temperature and length of time required depends on the type of enzymes added to the mixture, the rate of enzyme catalysis and the amount of the pomegranate solids contained in the mixture.

A combination of pectinase, cellulase and hemicellulase enzymes are added to the mixture, which is heated to a temperature of about 60° F. to about 210° F., and preferably about 110° F. to about 160° F., and more preferably of about 120° F. The mixture can be maintained at these temperatures, preferably with agitation or stirring, for about 45 to about 195 minutes, preferably for about 45 to about 75 minutes, and more preferably for about 60 minutes.

After the enzymes have at least partially degraded the pomegranate solids, the residual insoluble solid materials can be removed from the mixture. Optionally, a clarification agent, such as, but not limited to, bentonite, which can be used to remove proteins from the mixture, can be added before the step of removing the residual insoluble materials from the mixture. The removal of residual insoluble materials from the mixture can be accomplished by filtration, centrifugation, chromatographic techniques, or other techniques. Filtration techniques suitable for the practice of the methods disclosed herein include ultra-filtration at a molecular weight cut-off of at least 1,000 Da, preferably of about 4,500 Da, and more preferably of about 5,500 Da. In addition, micro-filtration, with a pore size of from 0.1 to 0.2μ, or to 0.5μ (i.e., a molecular cutoff of over 500,000 Da) may be used instead.

The resulting liquid extract can be concentrated in an evaporator under vacuum to about 50 to about 90 Brix (Bx), preferably to about 60 to about 80 Bx, more preferably to about 65 to about 70 Bx. The liquid extract can be concentrated to about 65 Bx. The extract can then be pasteurized at a temperature and for a length of time sufficient to kill any microorganisms that could cause disease, spoilage or undesired fermentation. Pasteurization of the extract can take place at a temperature of about 140° F. to about 280° F., preferably of about 195° F. to about 240° F., and more preferably of about 205° F. The pasteurization can also denature the remaining enzymes that were added to the mixture.

The extract from pomegranate can also be an extract from the whole pomegranate fruit or from any constituents of pomegranate fruit. Examples of constituents of pomegranate fruit that can be used to make the extract include, but are not limited to, juice, seed, and the inner and outer peel of pomegranate fruit. The extract is the juice extract of whole pomegranate fruit or can be from the inner or outer peel of pomegranate fruit. More than one extract can be mixed. (e.g., extracts of the whole pomegranate or any constituents of pomegranate).

Another method of making the extract includes the steps of crushing and squeezing the whole fruits of the pomegranate, including the inner and outer peels and the seeds, to yield a juice component and a first insoluble byproduct component and separating the juice component from the insoluble byproduct component. The juice component can be used as a juice extract. Alternatively, the insoluble byproduct component can be resuspended in an aqueous medium, such as, but not limited to, water or alcohol, and further crushed, squeezed, and mixed to yield a second soluble portion and a second insoluble byproduct component. The soluble portion can be separated from the second insoluble by product component to produce a second form of the extract. Alternatively, the soluble portion can be combined with the juice extract to produce a third form of extract.

In addition, the whole fruit of the pomegranate can be enzymatically treated to improve extraction and filtration. For example, pectinase can be used to treat the whole fruit to prevent the formation of pectin gels. Other enzymes known in the art can also be used as long as they can improve extraction and filtration of the extract.

The extract of pomegranate obtained by the methods outlined above can be in a liquid or solid form. A solid form of the extract can be made by lyophilizing the liquid extract. Alternatively, the constituents of the pomegranate, such as seeds, inner or outer peels, or any insoluble byproduct component discussed above, can be processed directly to form the solid form of the extract. For example, the constituents of the pomegranate can be dried, and processed into powder or pill forms to be used directly as the solid form of the extract.

Another alternative method of preparing a pomegranate extract polyphenols are recovered from the extract concentrate using FDA food-grade resins. Extract concentrate, diluted with water, can be passed through resin columns which preferentially adsorbs polyphenols minus most of the anthocyanins from the extract liquid. The resins do not chemically modify the polyphenols; they reversibly adsorb and desorb the polyphenols with their original chemical structure remaining unchanged. Non-phenol compounds, such as sugars, organic acids, cellulose, and other carbohydrates, can pass unadsorbed through the resin column. Polyphenols adsorbed on the resin can be recovered (de-adsorbed) using ethanol in water.

The recovered polyphenols can be concentrated by removing ethanol. The remaining polyphenol water solution can be dried to produce a pomegranate food polyphenol extract powder. The separation medium can include a synthetic polymeric adsorbent resin. Generally, these synthetic polymeric adsorbents take the form of non-ionic macroreticular resins that adsorb and release ionic and polar molecules (compounds) through hydrophobic and polar interactions; these are usually employed under isocratic conditions (i.e., only a single eluent of fixed composition is used). Such polymeric resins are usually derived from a synthetic hydrophobic polyaromatic resin such as cross-linked polyvinylbenzene (polystyrene) and polydivinylbenzene. These resins are manufactured under trade names such as Amberchrom™, Amberlite™, Diaion™, and Dowex™. One advantage of the polystyrene-divinylbenzene copolymer resin is the polyphenols are well adsorbed when dissolved in water or dilute aqueous C₁-C₃ alkanol (e.g., 2% v/v ethanol), preferably at the nominal operating range of 100° to 140° F.

It may also be possible to use natural polymeric media, such as microparticulate cellulose which is suitable for the separation of nucleotides, sugars, amino acids and polyphenols. Potential drawbacks to the use of microparticulate cellulose or derivatives thereof, include swelling in an aqueous environment and/or compressibility under pressure.

Other alternatives include, but are not limited to, dextran polymers (e.g, Sephadex™, Pharmacia UK) or agarose beads (e.g., Sepharose™, Pharmacia, UK). In one or more embodiments, the temperature range for operating the separation process can be in the range of 100° to 140° F. The separation medium can also be used at the temperature below 100° F., which can alter the adsorption characteristics of polyphenols and other phytochemicals of interest. In one or more embodiments, the flow rate through the separation medium can be in the range one to three bed volumes an hour, optimally two bed volumes per hour. A bed volume is the amount of the adsorbent resin in a separation medium. The volume and total time of flow of a particular feed stream into the column can be controlled by the desired output and the input stream. The optimal flow rate enables the separation medium to sequester polyphenols and other phytochemicals of interest, and rinse out the unbound material.

For the rinsing step in one or more embodiments, a dilute aqueous alcohol can be passed through the separation medium to remove unbound material (e.g., sugars, proteins, fibers, enzymes, carbohydrates). When obtaining a high purity of polyphenols in the pomegranate dry composition is desired, this rinsing step can be performed prior to the elution of polyphenols. Optionally, the rinse step may include back flushing the separation medium with a dilute aqueous alcohol to remove any insoluble material that may collect on the top of the separation medium. Dilute aqueous alcohol is any aqueous solution containing alkanol having one to four carbon atoms and of less than about 5% v/v, more preferable 2% v/v. Ethanol is a preferred alkanol since it is approved for food use, although other alkanols may be used. Water can also be employed, though it is less effective at getting unbound material out of the separation medium and reduces the purity of polyphenols in the pomegranate dry composition.

In one or more embodiments, the pomegranate extract is produced by obtaining pomegranate solids, which generally comprise the pericarp, the inner membrane and seed of the pomegranate. In one or more exemplary embodiments, the pomegranate solids are obtained and collected after the primary juice from the arils is substantially expelled or otherwise removed from the pomegranate by pressing, crushing, or other methods known to the art for extracting pomegranate juice. The pomegranate solids can then be transferred to mills (such as those sold by Reitz Mills) with ⅜ inch screens. The pomegranate solids can be milled to a fine puree and heated to approximately 125° F.

This step, coupled with the following enzyme addition, assists in breaking down the colloidal structure of the remaining pomegranate solids, thereby releasing the remaining soluble solids. The mixture can be heated to a temperature of about 125° F. for about two hours. In one or more of the exemplary embodiments described here three enzymes can be added to the mixture: pectinase (Rohapect® DA6L), cellulase/pectinase (Rohapect® CL), and hemi-cellulase/pectinase (Rohapect® B1L). These enzymes can liberate the remaining pomegranate soluble solids, such as sugars, minerals, anthocyanins, and remaining polyphenols. The mixture can then be pumped from the extraction plant to the primary processing plant where it is held in mash treatment tanks for approximately one hour. After one hour, about 50-100 pounds (or other appropriate amount) of bentonite in a 125 gallon water slurry, per 8,000 gallons of the mixture, is added for protein removal. The treated mixture can then be passed through a Westphalia 755 Decanter for removal of solids. The residual insoluble material is typically discharged as waste. The liquid extract then exits the decanter and is filtered on microfiltration membranes (such as one sold by Koch SUPER-COR®) at a 500,000 Da molecular weight cut-off and then filtered again on ultrafiltration membranes (such as those sold by Koch) at a 100,000 or 200,000 Da molecular weight cut-off. The filtered liquid extract is then applied to a rising-film plate evaporator (such as that sold by Schmidt-Bretten). Initial heat on this step is about 140° F. In this step, the filtered liquid extract is concentrated to about 15- to 20-Bx. The filtered liquid extract is maintained at the temperature of about 140° F. and then passed through a pre-heated preparative column at about 140° F. (e.g., 4-foot diameter, 4-foot tall) packed with an ion exchange resin, such as Amberlite™ FPX66 (Rohm and Haas, Philadelphia, Pa.) at the flow rate of about two bed volumes an hour until the resins gets loaded. Any portions of liquid effluent indicating a bleed-through of polyphenols may be collected for subsequent loading step. After the load step, dilute aqueous alcohol (2% ethanol/H₂O) can be passed through the preparative column at the flow rate of about two bed volumes an hour to remove unbound material. Dilute aqueous alcohol effluent is discarded as a waste. After the rinse step, concentrated aqueous alcohol (20% ethanol/H₂O) is applied to the resin and the liquid eluate containing polyphenols is collected.

Alternatively, a pomegranate extract concentrate can be prepared from fresh pomegranates crushed in a champagne press. Because whole fruit is crushed, beneficial antioxidants from the peel and membranes are added to those already found in the juice. After crushing, the juice can be enzymatically treated with pectinase, filtered, concentrated and stored at about −18° C.

Using the methods described above, pomegranate extracts containing phytochemicals can be prepared. Such extracts are characterized by a significantly higher total polyphenol content, particularly of the high molecular weight polyphenol (e.g., punicalagin), than is found in pomegranate juice. Such extracts can be obtained from the methods disclosed herein or by other methods. Extracts containing phytochemicals, polyphenols, punicalagin, punicalin, ellagic acid, and metabolites thereof are provided.

The extract can have a higher total polyphenol content, as compared to the pomegranate juice alone, but it also provides the broad spectrum of the different polyphenols found in pomegranates. The extract can comprise lower molecular weight polyphenols (e.g., anthocyanins) and the higher molecular weight polyphenols (e.g., punicalagin, punicalin, ellagic acid glycosides, ellagic acid polyphenols, ellagitannin and other hydrolysable tannins, such as punicacortein A, pedunculagin, and gallotanin dimers and trimers). The pomegranate extract can contain from about 30 μmols to about 3000 μmols per dosage unit of polyphenols.

Turning to the treatment of erectile dysfunction in men (and sexual dysfunction in women), compositions and methods are provided for ameliorating these dysfunctions in a subject. For example, a subject can be treated for erectile dysfunction by administering to the subject an effective amount of the pomegranate extract suitable in combination with an erectile dysfunction-treating compound. The pomegranate extract can be prepared by the methods discussed above, or by any method that allows for the highest level of polyphenol extraction from the pomegranates. The amount of pomegranate extract to be administered can be between about 1,000 mg to about 2,000 mg, or about 1,500 mg of the extract in solid form. Because of the synergistic activity between the pomegranate extract and the erectile dysfunction-treating compound, however, the dosage of the pomegranate extract can be reduced to about 500 mg to about 1,000 mg per dose. In one embodiment, the composition can contain from about 85% to about 95% total polyphenols. The erectile dysfunction-treating compound of the composition can be, but is not limited to vardenafil, tadalafil, and sildenafil.

The composition of the invention can comprise vardenafil in the approved dosages, which range from about 2.5 mg to about 20 mg (i.e., 2.5 mg, 5 mg, 10 mg, and 20 mg dosage forms). The maximum recommended dosing frequency is once per day about 60 minutes before sexual activity. Because of the enhanced, or synergistic, activity between vardenafil and the pomegranate extract, however, the dosage of vardenafil can be reduced to about 625 μg to about 2.5 mg per dose.

The composition of the invention can comprise tadalafil in the approved dosages, which range from about 2.5 mg to about 20 mg (i.e., 2.5 mg, 5 mg, 10 mg, and 20 mg dosage forms). Tadalafil has been approved by the FDA for once daily use at daily dosage of about 2.5 mg. Because of the synergistic activity between tadalafil and the pomegranate extract, however the dosage of tadalafil can be reduced to about 625 μg to about 2.5 mg per dose.

The composition of the invention can comprise sildenafil in the approved dosages, which range from about 20 mg to about 100 mg (i.e. 20 mg, 25 mg, 50 mg, and 100 mg dosage forms). The maximum recommended dosing frequency is once per day about 60 minutes before sexual activity. Because of the synergistic activity between sildenafil and the pomegranate extract, however, the dosage of sildenafil can be reduced to about 5 mg to about 20 mg per dose.

Each of the three erectile dysfunction-treating compounds described above has a similar range of side effects. Such effects include headaches, flushing, dyspepsia, nasal congestion, urinary tract infection, abnormal vision, diarrhea, dizziness, rash and back pain. Other more serious side effects include loss of hearing, priapism, and respiratory infection. A surprising and unexpected synergy between the pomegranate extract and the erectile dysfunction-treating compound can allow for a lower dose to be administered to a subject, which in turn, means that the likelihood and severity of side effects is substantially prevented or at least reduced in frequency and/or severity.

The inventors discovered the surprising and unexpected aspect of the present invention that pomegranate extract and an erectile dysfunction-treating compound act in an additive manner, and preferably synergistically in the treatment of ED, as well as acting as an antioxidant therapy for the oxidative stress important for the etiology of ED. Therefore, the combination of pomegranate extract and an erectile dysfunction-treating compound can also be useful for preventing smooth muscle dysfunction and fibrosis in ED.

The extract can be formulated with erectile dysfunction-treating compounds into various compositions. Compositions of the present invention can be in convenient dosage forms, including, but not limited to, tablets, suspensions, implants, solutions, emulsions, capsules, powders, syrups, liquid compositions, ointments, lotions, creams, pastes, gels. The composition can also be administered parenterally as injectable dosages in a physiologically acceptable carrier.

Compositions of the present invention can include a pharmaceutically acceptable excipient. Examples of pharmaceutically acceptable excipients include, but are not limited to, biocompatible vehicles, fillers, buffering agents, glidants, lubricants, adjuvants, additives, and/or diluents to achieve a composition usable as a dosage form. As used herein, the terms “pharmaceutically acceptable,” “physiologically tolerable,” and variations thereof, as they refer to compositions, carriers, diluents, and reagents, are used interchangeably and represent that the materials are capable of administration to or upon a mammal without the production of undesirable physiological effects.

Suitable carriers or excipients are inert ingredients and can be included in the composition. Excipients can include, but are not limited to, fillers, sugar alcohols, starch, lubricants, and binders. Examples of sugars include lactose, glucose, and sucrose. Sugar alcohols include mannitol, sorbitol, and xylitol. Examples of starch include wheat, corn, or potato starch, modified starch and sodium starch glycolate. Lubricants include talc, magnesium stearate, calcium stearate, colloidal silica, and stearic acid. Binders include polyvinylpyrrolidone, cellulose derivatives, carboxymethyl cellulose, hydroxylpropyl cellulose, hydroxypropylmethyl cellulose, methyl cellulose, and gelatin.

A pharmaceutical composition of the present invention can be administered orally to a subject in a single dose or multiple doses over a period of time generally by oral administration. However, parenteral, subcutaneous, intravenous, intramuscular, and interperitoneal injections can also be used to administer the composition. Various administration patterns will be apparent to those skilled in the art.

The dosage ranges for the administration of the compositions of the present invention are those large enough to produce the desired therapeutic effect. The dosage should not be so large as to cause any adverse side effects, such as unwanted cross-reactions. Generally, the dosage can vary with the age, weight, sex, condition, and extent of a condition in a subject, and the intended purpose. The dosage can be adjusted in the event of any counter indications, tolerance, or similar conditions. Those of skill in the art can readily evaluate such factors and, based on this information, determine the particular effective concentration of a composition of the present invention to be used for an intended purpose.

### Example 1

An exemplary analysis of the dry pomegranate composition now follows. The pomegranate dry composition was analyzed using high performance liquid chromatography (HPLC) and matrix-assisted laser desorption/ionization-time of flight (MALDI-TOF) mass spectrometry. The pomegranate dry composition has a higher proportional content of pomegranate polyphenols, primarily punicalagin and its tautomers. See Table 1 for breakdown of polyphenols in 100 grams of the pomegranate dry composition powder.

One created the powder and liquid forms provide similar polyphenol components of one serving of pomegranate juice. One serving of pomegranate juice contains at least 800 mg total natural polyphenols (650 mg gallic acid equivalent, GAE) with expected variation from year to year and batch to batch. Pomegranate juice has been shown to have up to 4,370 mg/L or 1,049 mg/8 oz of punicalagin compounds by Cerda et. al., 2004. GAE underestimates the total polyphenol level because gallic acid is not optimized standard. A 1,000 mg capsule of powder made using the process described herein contains at least 800 mg natural polyphenol using a pomegranate polyphenol standard. Table 2 below is illustrative: About 1000 mg of the dry pomegranate composition and about 200 mg of maltodextrin, which is used as a flow agent, are intimately mixed for prior to filling in a capsule comprising of hydroxypropyl methylcellulose.

This amount provides the polyphenol content equivalent to an 8 oz. bottle of pomegranate juice. The purification process of Example 1 enables the requisite polyphenol dose in a single dosage form, that may also contain the erectile dysfunction-treating compound.

TABLE 2
SPECIFICATIONS

| | |
|---|---|
| Chemical Classification | Organic, Nutritive |
| Physical Classification | Dried Fruit |
| Color | Red-Brown |
| Odor | Characteristic Tannin |
| Taste | Characteristic Tannin |
| Plant Part Used | Husk, Arils (Juice), |
| | and Fruit |

1.1.1 Chemical Parameters

| | |
|---|---|
| Total Phenolics | >85% (UV adsorption |
| | std. to pomegranate |
| | polyphenols) |
| pH (1 g/100 ml water) | 3.0 to 5.0 |
| Heavy Metals (ppm) | Less than 1 ppm. |

1.1.2 Physical Parameters

| | |
|---|---|
| Particle Size (wt. % | Less than 2%. |
| retained on 60 mesh) | |
| Bulk Density (g/cc) | 0.8 to 0.9 |
| Tap Density (g/cc) | 0.9 to 1.0 |

Microbiological Assays:

| | |
|---|---|
| Total Plate Count (CFU/g) | <1,000 |
| Yeast (CFU/g) | <100 |
| Mold (CFU/g) | <10 |
| Total Coliforms (CFU/g) | <10 (none detected) |
| E. coli (CFU/g) | <10 (none detected) |
| Salmonella (CFU/g) | Negative in 25 grams. |
| Staph. aureus (CFU/g) | <10 (none detected) |

SHELF LIFE

18 months at or below 70 F. in a sealed container.

2.1.1.1.1 PACKAGING

10 kg. in a double-lined plastic bag. Product should be stored
in the original sealed container and tightly closed after usage.

TABLE 3
Polyphenols in the pomegranate fruit polyphenol composition

| | Pomegranate Fruit Polyphenol Composition | | |
|---|---|---|---|
| | mg/1,000 mg | | |
| | Batch 1 | Batch 2 | Batch 3 |
| Anthocyanins | trace | trace | Trace |
| Gallagyl-type | 169.4 | 141.4 | 161.1 |
| (punicalagins & punicalin) | | | |
| Ellagic acid and derivatives | 27.6 | 33.1 | 35.9 |
| Other hydrolysable tannins | 593.0 | 665.5 | 663.1 |
| Total | 790.0 | 840.0 | 860.0 |

### Example 2

A randomized, placebo-controlled, double-blind, parallel design trial to evaluate the safety and efficacy of POM Wonderful pomegranate extract (pomegranate extract) capsules in male subjects with moderate to severe erectile dysfunction will be performed, and is expected to begin shortly. The primary objective of the study will be to evaluate the efficacy of two doses of pomegranate extract capsules in men with moderate to severe erectile dysfunction (ED) using a combination of diary-based and validated questionnaire endpoints. The secondary objective will be to characterize the pharmacologic profile of two doses of pomegranate extract capsules in men with moderate to severe ED using multiple validated endpoints. The safety objective will be to evaluate the safety profile of two doses of pomegranate extract capsules compared to placebo in men with moderate to severe ED.

This will be a multi-center, randomized, double-blind, placebo-controlled, parallel design study to examine the efficacy of pomegranate extract capsules versus placebo in restoring erectile function in male subjects with moderate to severe ED. After completion of an screening visit, each study subject will be issued a Sexual Encounter Profile (SEP) and asked to complete it during the four weeks prior to randomization. Subjects will be randomized to receive: 1 pomegranate extract and 1 placebo capsule per day for 12 weeks of active treatment; 2 pomegranate extract capsules per day for 12 weeks of active treatment; or 2 placebo capsules per day for 12 weeks of active treatment. The International Index of Erectile Function (IIEF), the Sexual Encounter Profile (SEP), and a Global Assessment Questionnaire will be administered at baseline, and then again at Days 28, 56, and 84 to evaluate response to treatment.

In order to be included in the upcoming study, the subjects must meet the following criteria:
Male 21 to 70 years old;
Erectile Function domain score of 10-19 on the International Index of Erectile Function (IIEF);
SEP question 3 answered “no” at least 50% of the time during the screening period;
In a stable, monogamous relationship with a consenting female partner and willing to attempt vaginal sexual intercourse on at least one occasion per week on average during each study period;
History of ED (clinically defined as the inability to attain and maintain an erection of the penis sufficient to permit satisfactory sexual intercourse) of at least 3 months duration;
Treated previously with a PDE-5 inhibitor with a satisfactory response, as defined by the patient; and
Must sign an informed consent.

The presence of any of the following will exclude a subject from study enrollment:
ED caused by untreated endocrine disease, i.e., hypopituitarism, hypothyroidism, hypogonadism;
A diagnosis of situational psychogenic ED;
Significant penile pathology, including but not limited to curvature, fibrosis, sexually transmitted disease, and penile implant;
Clinically significant hepatic, renal, neurological disease, diabetes mellitus, spinal cord injury, significant coronary heart disease, significant neurological disease or hepatitis B and/or C;
A history of prostate cancer or prostate surgery other than a transurethral resection of the prostate;
A history of alcoholism within the previous 2 years;
Current consumer of three or more units of alcohol per day (1 unit is equivalent to 1 glass of wine, 1 pint of beer, or 1 shot of hard liquor);
Taking ED therapy (prescription medications, over-the-counter medications, herbal preparations or medical devices) after the screening visit;
Participation in another study with an investigational drug or device during the 30 days prior to study entry; or
Have a condition interfering with his ability to provide informed consent or comply with study instructions.

The Study Product will be a powder, brown in color, encapsulated in a size 0 hard gelatin capsule. The capsule will be off-white in appearance. The Comparator Product will identical in weight and appearance to the Study Product

Investigational Products (Study and Comparator Products will be identical in appearance, with their identity blinded to the Investigator and subject) will be contained in blister packs packaged for weekly use. Identifiers will include: subject number; storage instructions and expiration, a notation that the product is for clinical trial use only; and that the product is manufactured for the sponsor. The study medication will be stored at room temperature, i.e., 20-25° C./68-77° F.).

Subjects will be randomly assigned to one of three treatment groups in a 1:1:1 randomization ratio: pomegranate extract 1 or 2 capsules or placebo. Randomization will be performed within each center in blocks of 6. The electronic case report form (eCRF) system will assign the blinded study medication by identification of the treatment group-specific kit codes to be dispensed.

There will be three co-primary efficacy variables, which will be: 1) Erectile Function (EF) domain score of the IIEF; 2) Q2 of the SEP, 3) Q3 of the on the SEP. The IIEF will be administered in the office at Visit 1 (Screening), Visit 2 (Day 1), Visit 3 (Day 28), Visit 4 (Day 56), and Visit 5 (Day 84). The SEP will be a diary measure completed by the subject after each sexual attempt.

Secondary efficacy variables will be total score on SEP and other domain scores of the IIEF, representing intercourse satisfaction (questions 6-8); orgasmic function (questions 9-10); overall satisfaction (questions 11-12), and responses to the Global Assessment Questionnaire.

In addition, other systemic markers of oxidative stress, including, e.g., serum lipid peroxidation by the thiobarbituric acid reactive substances (TBARS) and lipid peroxide assay, inflammation by HS c-reactive protein, and serum nitric oxide, will be explored during the course of the study.

Safety variables will be clinical laboratory, physical examination, electrocardiogram and adverse events.

The clinical data will be generated from blood samples for the assessment of clinical laboratory safety will be obtained at several visits. Clinical laboratory safety analyses will include:

1) Biochemistry: Glucose, calcium, albumin, total protein, sodium, potassium, bicarbonate, chloride, blood urea nitrogen, creatinine, alkaline phosphatase, alanine amino transferase, aspartate amino transferase, bilirubin, HS C-reactive protein and nitric oxide;

2) Hematology: Complete blood count (hemoglobin, hematocrit, red blood cell count, white blood cell count, white blood cell differential count, platelet count);

3) Urinalysis: Complete urinalysis; and

4) Additional tests will be analyzed at each visit for oxidative state and inflammation (lipid peroxidation), as well as urinary urolithins.

Adverse events will also be tracked. An adverse event will be any untoward medical occurrence in a subject participating in a clinical trial. An adverse event will also be any unfavorable and unintended sign, symptom or disease temporally associated with the use of the study medication, whether or not considered related to the study medication. Adverse events will be collected from the start of treatment until 30 days following the final visit dose of study medication. Any events occurring prior to beginning treatment will be recorded on the medical history page with the event name and onset date and end date if not continuing. Pre-existing, known clinically significant conditions observed at screening will be recorded as medical history.

Adverse events will also be coded into one of two causality categories: unrelated and related events. An unrelated event will be one that clearly and incontrovertibly due only to extraneous causes, and does not meet criteria listed under possible or probable. An unrelated event will not follow a reasonable temporal sequence from administration, and will be produced by the subject's clinical state or by environmental factors or other therapies administered. A related event will be one that follows a reasonable temporal sequence from administration, but will also be produced by the subject's clinical state, environmental factors or other therapies administered. Such an event will also reappear upon rechallenge, and follow a known pattern of response to the investigational medicinal product.

The study will include three populations: the Safety Population, the Intent-to-Treat Population, and the Per-Protocol Population. The Safety Population, which will determine the safety of the composition, will include all subjects who will be randomized and receive at least one dose of the treatment. The Intent-to-Treat Population will include subjects who will be randomized, receive at least one dose of the treatment, and finish at least one IIEF questionnaire. Last observation carried forward (LOCF) will be applied for any missing primary efficacy endpoints. This population will likely not complete the study. The Per-Protocol Population will include those subjects that complete the study. These subjects will be randomized, receive the correct randomized treatment assignment, finish all study visits, and fulfill all inclusion and exclusion criteria. Subjects that will be randomized into the study will be summarized by treatment group in terms of total number, number of completed and discontinued, and the reasons for study discontinuation.

The primary efficacy analysis will be performed. Changes from baseline of the primary efficacy endpoints will be summarized by treatment group. The statistical comparison between the active treatment groups and placebo will be performed using the analysis of variance (ANOVA) method. The outcome from ANOVA will be the primary analysis. The outcome adjusting for baseline value and study site interaction will be examined by fitting an analysis of covariance (ANCOVA) model, including study site, treatment, and the interaction term of study site by treatment, and the baseline measurement as covariates. A similar statistical analysis will be applied to the secondary efficacy endpoints. Safety will be assessed by evaluation of adverse events and clinical laboratory results.

### Example 3

A second series of studies to assess the efficacy of the pomegranate extract in combination with an erectile dysfunction-treating compound will be conducted using the same general protocol as described in Example 1. However, the investigational product will contain the pomegranate extract and an erectile dysfunction-treating compound at various concentrations. The erectile dysfunction-treating compound will be one or more of vardenafil, tadalafil, and sildenafil. This experiment will encompass dosages that will be used to demonstrate the potential synergy between the pomegranate extract and three erectile dysfunction-treating compounds. Tables 4-6 show a proposed dosage grid for testing different dosages of the pomegranate extract versus different dosages of each of three erectile dysfunction-treating compounds. Table 4 shows a proposed dosage of between 500 mg and 2,000 mg of the pomegranate extract in combination with one of four proposed doses of vardenafil ranging from one fourth of the lowest prescribed dose to the lowest prescribed dose. Table 5 shows the same proposed pomegranate extract doses in combination with one of four proposed doses of tadalafil ranging from one fourth of the lowest prescribed dose to the lowest prescribed dose. Table 6 again shows the same proposed pomegranate extract doses in combination with one of four proposed doses of sildenafil ranging from one fourth of the lowest prescribed dose to the lowest prescribed dose.

TABLE 4
Vardenafil

| | Pomegranate Extract Dosage (Across) | | | |
|---|---|---|---|---|
| | 500 mg | 1000 mg | 1500 mg | 2000 mg |
| Vardenafil Dosage (Down) | 625 μg | 625 μg | 625 μg | 625 μg |
| | 1250 μg | 1250 μg | 1250 μg | 1250 μg |
| | 1875 μg | 1875 μg | 1875 μg | 1875 μg |
| | 2500 μg | 2500 μg | 2500 μg | 2500 μg |

TABLE 5
Tadalafil

| | Pomegranate Extract Dosage (Across) | | | |
|---|---|---|---|---|
| | 500 mg | 1000 mg | 1500 mg | 2000 mg |
| Tadalafil Dosage (Down) | 625 μg | 625 μg | 625 μg | 625 μg |
| | 1250 μg | 1250 μg | 1250 μg | 1250 μg |
| | 1875 μg | 1875 μg | 1875 μg | 1875 μg |
| | 2500 μg | 2500 μg | 2500 μg | 2500 μg |

TABLE 6
Sildenafil

| | Pomegranate Extract Dosage (Across) | | | |
|---|---|---|---|---|
| | 500 mg | 1000 mg | 1500 mg | 2000 mg |
| Sildenafil Dosage (Down) | 5 mg | 5 mg | 5 mg | 5 mg |
| | 10 mg | 10 mg | 10 mg | 10 mg |
| | 15 mg | 15 mg | 15 mg | 15 mg |
| | 20 mg | 20 mg | 20 mg | 20 mg |

While various embodiments of the present invention have been described above, it should be understood that such disclosures have been presented by way of example only, and are not limiting. Thus, the breadth and scope of the present invention should not be limited by any of the above-described exemplary embodiments, but should be defined only in accordance with the following claims and their equivalents.

Having now fully described the invention, it will be understood by those of ordinary skill in the art that the same can be performed within a wide and equivalent range of conditions, formulations and other parameters without affecting the scope of the invention or any embodiment thereof.

1. A pharmaceutical composition comprising a combination of:
a) a therapeutically effective amount of a pomegranate extract; and
b) a therapeutically effective amount of an erectile dysfunction-treating compound, wherein the pharmaceutical composition further comprises at least one pharmaceutically acceptable excipient, wherein the combination has a synergistic effect on erectile dysfunction in a subject.

2. (canceled)

3. The pharmaceutical composition of claim 1, wherein the pomegranate extract comprises:
1) between about 20% to about 40% by weight comprises a combination of punicalin and punicalagin; and
2) between about 1% to about 5% by weight of ellagic acid.

4. The pharmaceutical composition according to claim 3, wherein the punicalagin comprises about 20% to about 25% punicalagin-α by weight of the composition and about 4% to about 5% punicalagin-β.

5. (canceled)

6. The pharmaceutical composition according to claim 1, wherein the amount of the erectile dysfunction-treating compound required for therapeutic efficacy is lower in combination with the pomegranate extract than alone.

7-8. (canceled)

9. The pharmaceutical composition according to claim 1, wherein the composition is administered orally, by injection, transdermally, buccally, or topically.

10. The pharmaceutical composition according to claim 1, wherein the composition is an immediate release composition, a delayed-release composition, an extended-release composition, a mixed-release composition, or an enterically-coated composition.

11-12. (canceled)

13. The pharmaceutical composition according to claim 1, further comprising a polyphenol selected from the group consisting of gallic acid, an isoflavone, an anthocyanin, a hydrolyzable tannin, and combinations thereof.

14. A package comprising 28 doses of the pharmaceutical composition of:
a. therapeutically effective amount of a pomegranate extract; and
b. a therapeutically effective amount of an erectile dysfunction-treating compound, wherein the pharmaceutical composition further comprises at least one pharmaceutically acceptable excipient, wherein the combination has a synergistic effect on erectile dysfunction in a subject.

15. (canceled)

16. The package according to claim, 14 wherein the dosage of the erectile dysfunction-treating compound is reduced over the 28 doses.

17-22. (canceled)

23. A method of treating erectile dysfunction comprising:
a) administering to a subject a pharmaceutical composition comprising a combination of
i) a therapeutically effective amount of a pomegranate extract; and
ii) therapeutically effective amount of an erectile dysfunction-treating compound, wherein the pharmaceutical composition further comprises at least one pharmaceutically acceptable excipient, wherein the combination has a synergistic effect on erectile dysfunction in the subject.

24. (canceled)

25. The method according to claim 23, wherein the pomegranate extract comprises
1) between about 20% to about 40% by weight comprises a combination of punicalin and punicalagin; and
2) between about 1% to about 5% by weight of ellagic acid.

26. The method according to claim 23 wherein the punicalagin comprises about 20% to about 25% punicalagin-a by weight of the composition and about 4% to about 5% punicalagin-β.

27. (canceled)

28. The method according to claim 23, wherein the amount of amount of the erectile dysfunction-treating compound required for therapeutic efficacy is lower in combination with the pomegranate extract than alone.

29-30. (canceled)

31. The method according to claim 23 wherein the composition is administered orally, by injection, transdermally, buccally, or topically.

32. The method according to claim 23 wherein the composition is an immediate release composition, a delayed-release composition, an extended-release composition, a mixed-release composition, or an enterically-coated composition.

33-34. (canceled)

35. The method according to claim 23 further wherein the composition further comprises a polyphenol selected from the group consisting of gallic acid, an isoflavone, an anthocyanin, a hydrolyzable tannin, and combinations thereof.

36. The method according to claim 23 wherein the therapeutically effective amount of the erectile dysfunction-treating compound is reduced over a predetermined time period.

37-41. (canceled)

42. The method of claim 23 comprising
a) providing a package containing 28 dosages of the pharmaceutical composition.

43. (canceled)

44. The method according to claim 42, wherein the dosage of the erectile dysfunction-treating compound is reduced over the 28 doses.

45-46. (canceled)

47. The method according to claim 42, wherein the dosage of the pomegranate extract is reduced over the 28 doses.

48-50. (canceled)
